(19) United States
(12) Patent Application Publication
(10) Pub. No.: US 2010/0004514 A1
(43) Pub. Date: Jan. 7, 2010

(54) INTUBATION LARYNGOSCOPE WITH TWO-SIDED BLADE

(75) Inventors: Michael Shalman, Ashdod (IL); Leonid Lukov, Beer-Sheva (IL)

Correspondence Address: NIXON & VANDERHYE, PC, 901 NORTH GLEBE ROAD, 11TH FLOOR, ARLINGTON, VA, 22203, US

(73) Assignee: M.S. Vision LTD., Ashkelon (IL)

(21) Appl. No.: 12/312,539

(22) PCT Filed: Nov. 14, 2007
(86) PCT No.: PCT/IB2007/003499

§ 371 Date: May 15, 2009

(30) Foreign Application Priority Data
Nov. 15, 2006 (IL) 179291

(51) Int. Cl. A61B 1/267

(52) U.S. Cl. 600/187; 600/194

(57) ABSTRACT

Intubation laryngoscope comprises blade (201) including upper elongated part (206) and lower longitudinal part (211) disposed below upper elongated part (206) between its left (207) and right (209) edges. Lower longitudinal part (211) along with a right portion of upper elongated part (206) form right passageway (213), and lower longitudinal part (211) along with a left portion of upper elongated part (206) form left passageway (214). This allows both the right-handed operation using right-passageway (213) for the advancement of endotracheal tube, while left passageway (214) can be used for arrangement of an oxygenation tube, suction tube or endoscope probe, and the left-handed operation using left passageway (214) for the advancement of endotracheal tube, while right passageway (213) can be used for the arrangement of mentioned auxiliary means.

### BACKGROUND

1. Field of the Invention

The present invention relates to the laryngoscopes used for the intubation and diagnostic purposes having reusable or disposable blades adapted for right- and left-handed operation.

2. Prior Art

The usual intubation laryngoscope 100, for example fabricated by Welch Allyn company (see FIGS. 1, 2), and Heine company (see FIGS. 3, 4) comprises several detachable blades of curvilinear Macintosh profile of various size designed for operations with children and adults. Each blade 101 has blade distal end portion 102 designed to expose a tracheal entrance in order to insert an endotracheal tube therein, blade proximal end portion 103 designed for detachable connection with laryngoscope handle 104 through holder 105, upper elongated part 106 to be interacted with patient's tongue, and lower longitudinal part 107 disposed laterally and below relative to upper elongated part 106. Lower longitudinal part 107 functions as a strengthening element of blade 101. The position of lower longitudinal part 107, on the left or on the right side relative to upper longitudinal part 106, determines what operator's hand, right or left, respectively, is used for inserting the endotracheal tube, while another operator's hand holds handle 104. The all shown prior art versions (FIGS. 1 to 4) are designed for the right-handed operation because lower longitudinal part 107 is disposed on the left side of upper elongated part 106. At present, the special blades for left-handed operation are manufactured in very small proportion, so that many of the great hospitals have no the left-handed blades at all. Blade proximal end portion 103 is provided with fastening appliance 108 designed for detachable connecting blade 101 to holder 105 and comprising the proximal end of fiberoptic light guide 109. The illumination system of laryngoscope comprises an illumination lamp and batteries hosed inside handle 104, as well as mentioned fiberoptic light guide 109. The latter is extended distally of fastening appliance 108 and disposed on lower longitudinal part 107 so that its distal face 110 emitting an illumination light is located beneath upper elongated part 106. In the embodiment of company Heine, named Greenline-profile (FIGS. 3, 4), the fiberoptic light guide is housed in closed sheath 111 presenting the part of the lower longitudinal part 107. Thus, lower longitudinal part 107 also functions as a carrier of light guide 109. Besides, lower longitudinal part 107 is provided with lower lateral flange 112 designed to interact with patient's teeth and disposed on the side of part 107 opposite relative to upper elongated part 106. Such disposition of flange 112 increases the overall transversal dimension of blade 101.

The disadvantage of the existing laryngoscope blades is the incapability of both right-handed and left-handed operating. Typically, the blades are fabricated for the right-handed operation and are very inconvenient for left-handed anesthesiologists. Another disadvantage is the restricted visibility of the tracheal entrance during intubation, when the relatively narrow passageway defined with upper elongated part 106, lower longitudinal part 107 and inner side of patient's cheek is occupied with the endotracheal tube. With the availability of some patient's anatomical peculiarities this situation leads to inserting the endotracheal tube by anesthesiologist practically blindly. The identical situation also arises, when the mentioned passageway is occupied with tumor on patient's right cheek or the vision field is masked with bleeding from right cheek. Another disadvantage is the restricted capability of the introduction some additional means such as oxygenation tube, suction tube or endoscope probe into patient's mouth during the intubation process because of the insufficient space between the blade and patient's mouth walls.

### SUMMARY OF THE INVENTION

The objective of the present invention is providing the capability of both right-handed and left-handed operation of the laryngoscope.

Another objective is the substantial improvement of the observation of the tracheal entrance and the endotracheal tube during the intubation process.
Another objective is considerable easing the intubation execution in the case of pathological changes or trauma of mouth's right portions.

The above noted objectives are accomplished with an intubation laryngoscope comprising a handle and at least one detachable blade having a blade distal end portion designed to expose a tracheal entrance in order to insert an endothracheal tube therein, and a blade proximal end portion connected to the handle. The blade includes an upper elongated part to be interacted with patient's tongue, and a lower longitudinal part disposed at least partly below the upper elongated part between the left and right edges of the upper elongated part and extended distally from the blade proximal end portion. Therewith, the lower longitudinal part along with the right portion of the upper elongated part located to the right of the lower longitudinal part form a right passageway, and the lower longitudinal part along with the left portion of the upper elongated part located to the left of the lower longitudinal part form a left passageway.

In version embodiment, the passageways are substantially equal in size, allowing: both the right-handed operation using the right passageway for the advancement of endotracheal tube into tracheal entrance during the intubation procedure, while the left passageway can be used for the observation of the tracheal entrance and the process of inserting endotracheal tube therein or for arrangement of auxiliary means such as oxygenation tube, suction tube or endoscope probe,

and the left-handed operation using said left passageway for the advancement of endotracheal tube into tracheal entrance during the intubation procedure, while the right passageway can be used for the observation of the tracheal entrance and the process of inserting endotracheal tube therein or for the arrangement of auxiliary means such as oxygenation tube, suction tube or endoscope probe. Thus, the capability of both right-handed and left-handed operation of the laryngoscope is accomplished. The simultaneous presence of two passageways allows using one of them for the endotracheal tube insertion and second of them for the observation of the tracheal entrance and endotracheal tube during the intubation process. The second passageway also can be used for arrangement of auxiliary means such as oxygenation tube, suction tube or endoscope probe. In the case of substantial pathological changes or trauma of mouth's one side, the presence of two passageways also allows considerable easing the intubation execution due to choice for the intubation the passageway located on mouth's healthy side.

In another version embodiment, designed preferably for the right-handed operation, the right passageway has substantially more width than the left passageway, thereby preventing the blade from substantial increase of its overall transversal dimension and allowing the right-handed operation with using the right passageway for the advancement of endotracheal tube into tracheal entrance during the intubation procedure, while the relatively narrow left passageway can be used for the observation of the tracheal entrance and the process of inserting endotracheal tube therein or for arrangement of auxiliary means such as oxygenation tube, suction tube or endoscope probe.

In another version embodiment, designed preferably for the left-handed operation, the left passageway has substantially more width than the right passageway, thereby preventing the blade from substantial increase of its overall transversal dimension and allowing the left-handed operation with using the left passageway for the advancement of endotracheal tube into tracheal entrance during the intubation procedure, while the right passageway can be used for the observation of the tracheal entrance and the process of inserting endotracheal tube therein or for arrangement of auxiliary means such as oxygenation tube, suction tube or endoscope probe.

Two last version embodiments allow substantial improvement of the capabilities of the intubation process visualization and applying the auxiliary means without any blade widening.

The intubation laryngoscope comprises an illumination system designed for illuminating the zone of the tracheal entrance and including a light guide, which during the intubation process is at least partly disposed at the lower longitudinal part.

The lower longitudinal part includes a lower surface facing downward and adapted to contact with patient's upper teeth. Therewith, as distinct from the existing laryngoscopes, the lower surface does not increase the overall transversal dimension of the blade due to its disposition in the mid zone of the blade. As a result, the overall transversal dimension of the proposed blade is not increased considerably, except that the blade has two passageways.

The lower longitudinal part serves also as a strengthening element of the blade. Thus, the lower longitudinal part serves as a blade strengthening element, a light guide carrier and a blade member contacting with patient's teeth.

In version embodiment, the upper elongated part of the blade is provided with at least one lateral flange extended downward from a lateral edge of the upper elongated part and designed to strengthen the upper elongated part and prevent patient's tongue from entering one of the passageway. Specifically, the latter is important for the above blades with narrowed one of the passageway. Such lateral flange is relatively short and does not designed for interacting with patient's teeth.

In version embodiment, the lower longitudinal part is substantially shorter than the upper elongated part, and the lateral flange is disposed in the location zone of a distal end of the lower longitudinal part and distally of it.

### BRIEF DESCRIPTION OF THE DRAWINGS

FIGS. 1, 2 show the existing intubation laryngoscopes (Prior Art) of “Welch Allyn” company (USA), wherein:

FIG. 1 shows the intubation laryngoscope assembly.

FIG. 2 shows one of the blades of the intubation laryngoscope.

FIGS. 3, 4 show the existing intubation laryngoscopes (Prior Art) of “Heine” company (Germany), wherein:

FIG. 3 shows the intubation laryngoscope assembly.

FIG. 4 shows one of the blades of the intubation laryngoscope.

The drawings presented in the following relate to the present invention.

FIGS. 5, 6 show the intubation laryngoscope with reusable two-sided blades having traditional blade holder, fastening appliance and illuminating means, wherein:

FIG. 5 shows the intubation laryngoscope assembly.

FIG. 6 shows one of the two-sided blades of the intubation laryngoscope.

FIGS. 7 to 11 show the intubation laryngoscope with disposable plastic two-sided blades and single illuminating means completely mounted on the handle, wherein:

FIG. 7 shows the intubation laryngoscope assembly.

FIG. 8 shows the two-sided blade for medium adults.

FIG. 9 shows the two-sided blade for infants.

FIG. 10 shows the two-sided blade with non-equal width of left and right passageways and lateral flange.

FIG. 11 shows the two-sided blade for large adults having two lateral strengthening flanges.

### DETAILED DESCRIPTION OF THE INVENTION

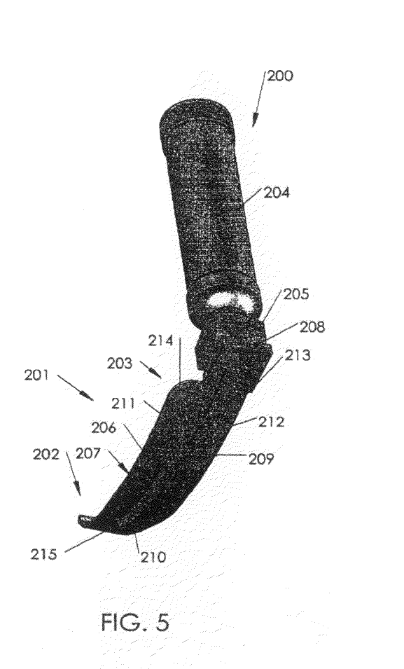

The explanation of the present invention is offered with references made to the attached drawings in FIGS. 5 to 11.

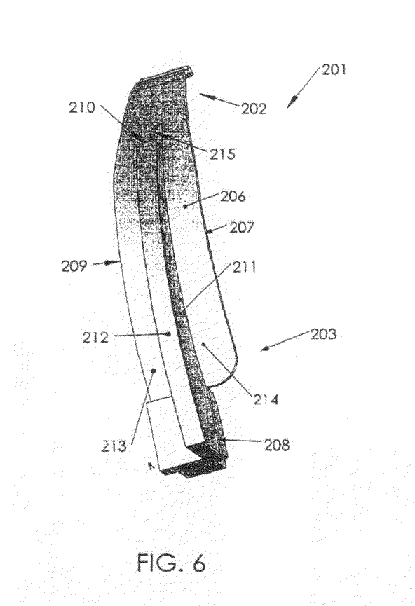

The drawings in FIGS. 5, 6 show intubation laryngoscope 200 comprising handle 204 and blade 201 having blade distal end portion 202 designed to expose the tracheal entrance in order to insert an endothracheal tube therein and blade proximal end portion 203. Blade 201 is provided with fastening appliance 208 detachably connecting blade 201 to holder 205 of handle 204. Blade 201 also includes upper elongated part 206 to be interacted with patient's tongue, lower longitudinal part 211 disposed below upper elongated part 206 between its left 207 and right 209 edges and extended distally from blade proximal end portion 203. Therewith, lower longitudinal part 211 along with a right portion of upper elongated part 206 located to the right of lower longitudinal part 211 form right passageway 213, and lower longitudinal part 211 along with a left portion of upper elongated part 206 located to the left of lower longitudinal part 211 form left passageway 214. Passageways 213 and 214 are substantially equal in size, allowing the right-handed operation using right passageway 213 for the advancement of endotracheal tube into tracheal entrance during the intubation procedure, while left passageway 214 can be used for the observation of the tracheal entrance and the process of inserting endotracheal tube therein or for arrangement of auxiliary means such as an oxygenation tube, suction tube or endoscope probe, as well as the left-handed operation using left passageway 214 for the advancement of endotracheal tube into tracheal entrance during the intubation procedure, while right passageway 213 can be used for the observation of the tracheal entrance and the process of inserting endotracheal tube therein or for the arrangement of auxiliary means such as oxygenation tube, suction tube or endoscope probe. Intubation laryngoscope 200 has an illumination system designed for illuminating the zone of a tracheal entrance during the intubation process and including batteries and illumination lamp housed in handle 204 and a light guide disposed inside lower longitudinal part 211 and having distal end 210 emitting illumination light. Lower longitudinal part also includes lower surface 212 facing downward and adapted to contact with patient's upper teeth. Therewith, as distinct from the existing laryngoscopes, lower surface 212 does not increase the overall transversal dimension of blade 201 due to its disposition in the mid zone of the blade. As a result, the overall transversal dimension of blade 201 is not considerably increased, except that the blade has two passageways. Thus, lower longitudinal part 211 serves as a carrier of the light guide, a member designed to contact with patient's upper teeth, as well as a blade strengthening element.

As a result, the simultaneous presence of two passageways on one blade provides the capability of both right-handed and left-handed operation of the laryngoscope. This also allows using one of two passageways for the endotracheal tube insertion and second of them for the observation of the tracheal entrance and endotracheal tube during the intubation process, thereby considerably improving the capability of the intubation process visualization. The second passageway also can be used for the arrangement of auxiliary means such as oxygenation tube, suction tube or endoscope probe. In the case of considerable pathological changes or trauma of mouth's one side, the presence of two passageways allows easing the intubation execution due to choice for the intubation the passageway located on mouth's healthy side.

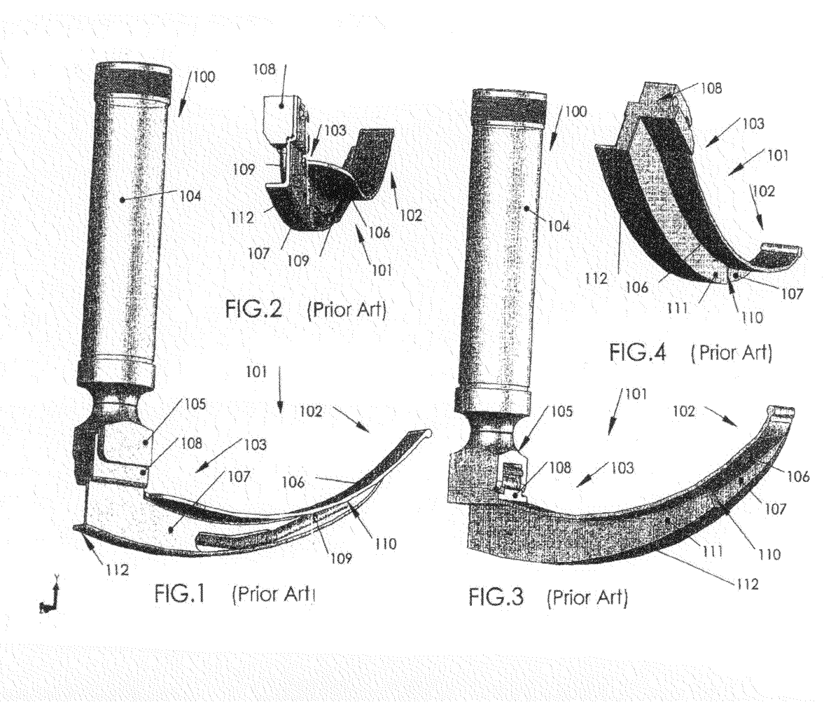

Laryngoscope 200, shown in FIGS. 5, 6, can be presented as a reusable laryngoscope made of metal and including several blades (as usual four blades) of different size designed for patients of various ages. Each of these blades is provided with its own lower longitudinal part 211 enough long to provide the required strength of blade 201 along its whole length. Distal end of lower longitudinal part 211 is terminated in rounded nose 215 to prevent patient's tissue from trauma during inserting blade 201 into patient's mouth. The design of fastening appliance 208, holder 205 and to great extent the illumination means are identical to Prior Art (see FIGS. 1, 2).

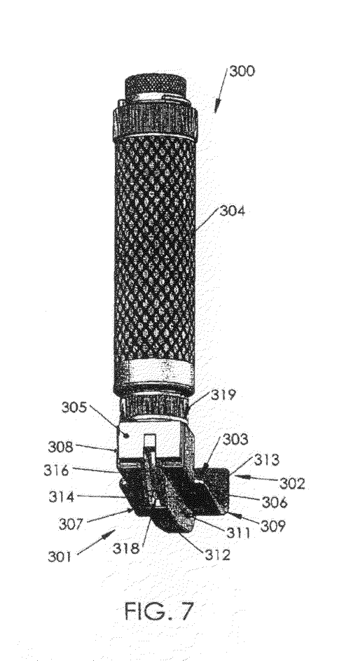

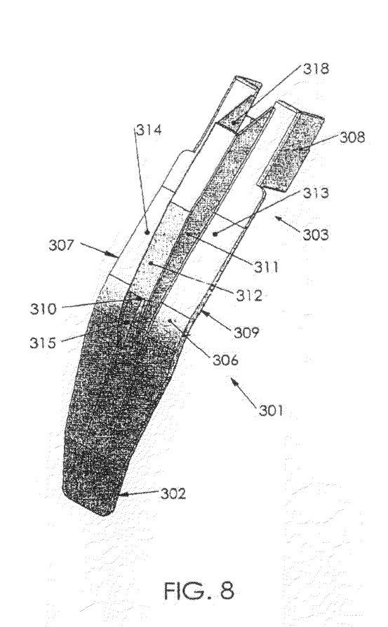

FIGS. 7 to 11 show the laryngoscope version 300 with disposable plastic blades 301, 317, 320, 324. The laryngoscopes 200 and 300 to a great extent are identical and the designations of their identical parts have the same two last numerals. As distinct from laryngoscope 200, laryngoscope 300 is provided with the illumination means with single light guide 316, common for all blades and having a distal portion inserted into blade socket 318, when blade fastening appliance 308 is connected to holder 305 and fixed there by nut 319. Blade socket 318 is disposed inside lower longitudinal part 311. Otherwise, blade 301 (see FIG. 8), designed for medium adults, have elements identical to above blade 201 including upper elongated part 306, equal right 313 and left 314 passageways, lower surface 312 to be contacted with patient's upper teeth, and end rounded noses 315. The functioning, operation and advantages of blades 201 and 301 are identical.

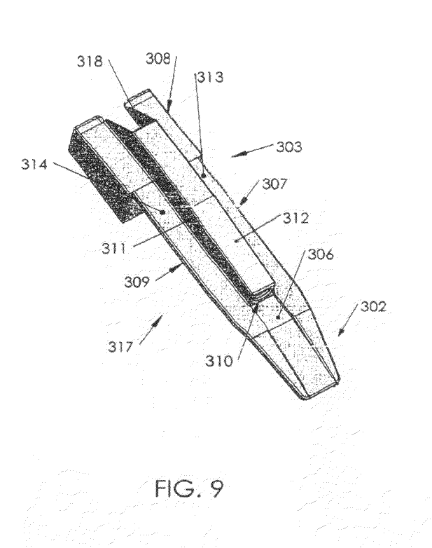

The peculiarity of plastic disposable blade 317 designed for infants (see FIG. 9) consists only in the design of distal end 310 of lower longitudinal part 311 deprived of rounded nose because of the short length of this blade. To prevent the traumatic act of end 310 onto patient's tissues it is partly buried in upper longitudinal part 306 and its edges are rounded.

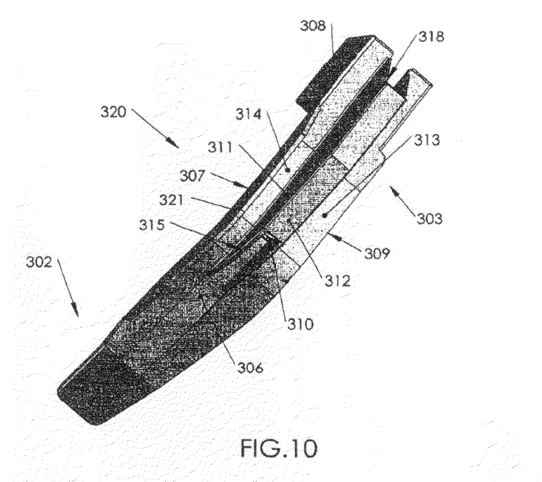

The disposable plastic blade 320 (see FIG. 10) designed for medium adults and to be used preferably for the right-handed operation, has right passageway 313 whose width is substantially more than one of left passageway 314. This prevents blade 320 from substantial increase of its overall transversal dimension and allows the right-handed operation with using right passageway 313 for the advancement of endotracheal tube into tracheal entrance during the intubation procedure, while left passageway 314 can be used for the observation of the tracheal entrance and the process of inserting endotracheal tube therein or for arrangement of auxiliary means such as oxygenation tube, suction tube or endoscope probe. Upper elongated part 306 is provided with one lateral flange 321 extended downward from lateral edge 307 of upper elongated part 306 to strengthen upper elongated part 306 and prevent patient's tongue from entering passageway 314. Therewith, the height of lateral flange 321 is restricted to prevent it from contacting with patient's upper teeth during the intubation process. The function of contacting with patient's upper teeth is completely fulfilled with lower surface 312 of lower longitudinal part 311. This version embodiment allows substantial improvement of the capabilities of the intubation process visualization and applying the auxiliary means without any blade widening.

In blade version embodiment (not shown), designed preferably for the left-handed operation, the left passageway has substantially more width than the right passageway, thereby preventing the blade from substantial increase of its overall transversal dimension and allowing the left-handed operation with using the left passageway for the advancement of endotracheal tube into tracheal entrance during the intubation procedure, while the right passageway can be used for the observation of the tracheal entrance and the process of inserting endotracheal tube therein or for arrangement of auxiliary means such as oxygenation tube, suction tube or endoscope probe. This blade version and previous blade 320 have practically the same advantages.

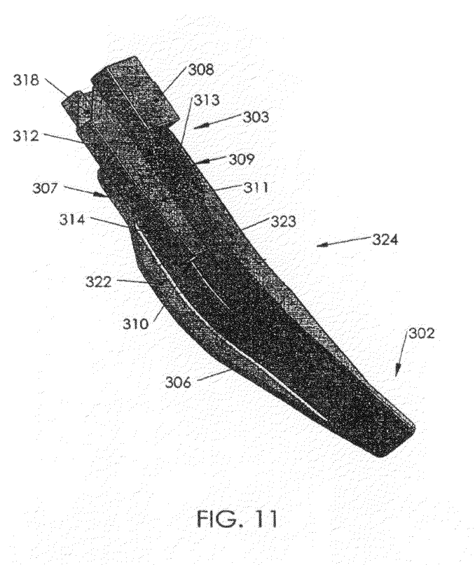

FIG. 11 shows disposable plastic blade 324 designed for large adults. In this case, the capability of lower longitudinal part 311 to provide the required strength of the distal portion of blade 324 is restricted because lower longitudinal part 311 is considerably shorter than upper elongated part 306. Therefore upper elongated part 306 is provided with two lateral flanges 322 and 323 disposed on the portion of part 306 including the location zone of a distal end 310 of lower longitudinal part 311 and distally of it. The disposition, design and restricted height of flanges 322, 323 do not allow their use for the operation contact with the patient's upper teeth.

1. An intubation laryngoscope comprising
a handle,
at least one blade, having
a blade distal end portion designed to expose a tracheal entrance in order to insert an endothracheal tube therein,
a blade proximal end portion connected to said handle,
an upper elongated part to be interacted with patient's tongue,
a lower longitudinal part disposed at least partly below said upper elongated part between left and right edges of said upper elongated part and extended distally from said blade proximal end portion, so that
said lower longitudinal part along with a right portion of said upper elongated part located to the right of said lower longitudinal part form a right passageway, and said lower longitudinal part along with a left portion of said upper elongated part located to the left of said lower longitudinal part form a left passageway,
therewith, at least one of said passageways is designed for the advancement of endotracheal tube into tracheal entrance during the intubation procedure.

2. The intubation laryngoscope of claim 1, wherein said passageways are substantially equal in size, allowing
both the right-handed operation using said right passageway for the advancement of endotracheal tube into tracheal entrance during the intubation procedure, while said left passageway can be used for the observation of the tracheal entrance and the process of inserting endotracheal tube therein or for arrangement of auxiliary means such as oxygenation tube, suction tube or endoscope probe,
and the left-handed operation using said left passageway for the advancement of endotracheal tube into tracheal entrance during the intubation procedure, while said right passageway can be used for the observation of the tracheal entrance and the process of inserting endotracheal tube therein or for the arrangement of auxiliary means such as oxygenation tube, suction tube or endoscope probe.

3. The intubation laryngoscope of claim 1, designed preferably for the right-handed operation, wherein said right passageway has substantially more width than said left passageway, thereby preventing said blade from substantial increase of its overall transversal dimension and allowing the right-handed operation with using said right passageway for the advancement of endotracheal tube into tracheal entrance during the intubation procedure, while said left passageway can be used for the observation of the tracheal entrance and the process of inserting endotracheal tube therein or for arrangement of auxiliary means such as oxygenation tube, suction tube or endoscope probe.

4. The intubation laryngoscope of claim 1, designed preferably for the left-handed operation, wherein said left passageway has substantially more width than said right passageway, thereby preventing said blade from substantial increase of its overall transversal dimension and allowing the left-handed operation with using said left passageway for the advancement of endotracheal tube into tracheal entrance during the intubation procedure, while said right passageway can be used for the observation of the tracheal entrance and the process of inserting endotracheal tube therein or for arrangement of auxiliary means such as oxygenation tube, suction tube or endoscope probe.

5. The intubation laryngoscope of claim 1, wherein there is an illumination system designed for illuminating the zone of a tracheal entrance during the intubation process and including at least one component, which during the intubation process is disposed at said lower longitudinal part.

6. The intubation laryngoscope of claim 5, wherein said an illumination system component is a light guide, which during the intubation process is at least partly disposed at said lower longitudinal part.

7. The intubation laryngoscope of claim 5, wherein said blade lower longitudinal part includes a blade socket located at said blade proximal end part and designed to insert therein said illumination means component common for all blades of a laryngoscope blade set.

8. The intubation laryngoscope of claim 5, wherein said lower longitudinal part has a lower surface facing downward and adapted to contact with patient's upper teeth.

9. The intubation laryngoscope of claim 8, wherein said lower longitudinal part serves as a blade strengthening element, a carrier of said illumination system component, and a member designed to contact with patient's upper teeth.

10. The intubation laryngoscope of claim 6, wherein said upper elongated part is provided with at least one lateral flange extended downward from a lateral edge of said upper elongated part and designed to strengthen said upper elongated part and prevent patient's tongue from entering one of said passageways, therewith the height of said lateral flange is restricted to prevent said lateral flange from contacting with patient's upper teeth during the intubation process.

11. The intubation laryngoscope of claim 10, wherein said lower longitudinal part is substantially shorter than said upper elongated part and said lateral flange is disposed on a restricted portion including the location zone of a distal end of said lower longitudinal part and distally of it.

12. The intubation laryngoscope of claim 11, wherein there are two said lateral flanges.
